(19) United States
(12) Patent Application Publication
(10) Pub. No.: US 2020/0265936 A1
(43) Pub. Date: Aug. 20, 2020

(54) HEALTH MANAGEMENT APPARATUS, METHOD FOR OPERATING HEALTH MANAGEMENT APPARATUS, AND PROGRAM FOR OPERATING HEALTH MANAGEMENT APPARATUS

(71) Applicant: FUJIFILM CORPORATION, Tokyo (JP)

(72) Inventors: Shigetoshi ISHIKAWA, Kanagawa (JP); Yasuhisa KANEKO, Kanagawa (JP); Haruyasu NAKATSUGAWA, Kanagawa (JP)

(21) Appl. No.: 16/776,520

(22) Filed: Jan. 30, 2020

(30) Foreign Application Priority Data
Feb. 18, 2019 (JP) 2019-026814

(51) Int. Cl. G16H 15/00; G16H 30/20; G16H 30/40; G06F 3/0482; G06F 3/0484; G06K 9/00

(52) U.S. Cl. CPC G16H 15/00; G16H 30/20; G16H 30/40; G06K 2209/051; G06F 3/04845; G06F 3/0484; G06K 9/00671; G06F 3/0482

(57) ABSTRACT

A CPU of a health management apparatus functions as a first acquisition unit, a second acquisition unit, and a screen output control unit. The first acquisition unit acquires medical examination results which are results of a medical examination of a pet. The second acquisition unit acquires an optical image of the pet. The screen output control unit controls an output of a medical examination result display screen including the medical examination results and at least one of the optical image or a schema diagram created on the basis of the optical image.

### CROSS-REFERENCE TO RELATED APPLICATIONS

The present application claims priority under 35 U.S.C. § 119 to Japanese Patent Application No. 2019-026814, filed on Feb. 18, 2019. The above application is hereby expressly incorporated by reference, in its entirety, into the present application.

### BACKGROUND

### 1. Technical Field

The technology of the present disclosure relates to a health management apparatus, a method for operating the health management apparatus, and a program for operating the health management apparatus.

### 2. Description of the Related Art

A medical examination that regularly performs various medical tests, such as a specimen examination and an image examination, is performed in order to manage the health condition of a subject. For example, JP5868536B discloses an aspect in which medical examination results are displayed together with a schema diagram showing the shape of a subject prepared in advance as an aspect in which the results of the medical examination (hereinafter, simply referred to as medical examination results) are displayed. JP5868536B discloses a person and a pet (animal) as subjects. In addition, JP5868536B discloses an aspect in which a schema diagram showing a part or the entire body of the pet may be used.

### SUMMARY

There are many breeds of pets, such as dogs and cats, and the shape of the pet varies depending on the breed. However, in JP5868536B, the schema diagram does not vary depending on the breed since it is prepared in advance. For this reason, it is considered that the schema is often different from the actual shape of the pet. In a case in which the schema diagram is different from the actual shape of the pet, a viewer, such as a pet owner, feels uncomfortable first. There is a concern that the sense of discomfort will interfere with the smooth understanding of the medical examination results.

An object of the technology of the present disclosure is to provide a health management apparatus that enables a user to smoothly understand the medical examination results of a pet, a method for operating the health management apparatus, and a program for operating the health management apparatus.

In order to achieve the object, according to the present disclosure, there is provided a health management apparatus comprising: a first acquisition unit that acquires medical examination results which are results of a medical examination of a pet; a second acquisition unit that acquires an optical image of the pet; and a screen output control unit that controls an output of a medical examination result display screen including the medical examination results and at least one of the optical image or a schema diagram created on the basis of the optical image.

Preferably, the health management apparatus further comprises a selection unit that selects an important item from items of the medical examination on the basis of selection information stored in advance. Preferably, the screen output control unit displays the important item in a display format different from a display format of other items on the medical examination result display screen.

Preferably, the selection information is information related to normal ranges of numerical values of the items and the selection unit selects an item whose numerical value is out of the normal range as the important item.

Preferably, the screen output control unit displays organs so as to be superimposed on least one of the optical image or the schema diagram and displays an annotation indicating an organ related to the important item on at least one of the optical image or the schema diagram.

Preferably, the medical examination includes an image examination and the screen output control unit displays a frame indicating an imaging region in the image examination so as to be superimposed on a part of a body of the pet drawn in at least one of the optical image or the schema diagram.

Preferably, the screen output control unit displays a medical image obtained by the image examination as the medical examination results in a region different from the frame so as to be enlarged and reduced and changes a display size of the frame in operative association with the enlargement and reduction of the medical image.

Preferably, the screen output control unit displays a medical image obtained by the image examination as the medical examination results in the frame and changes transparency of at least one of the optical image or the schema diagram in the frame in response to an operation of a user.

According to the present disclosure, there is provided a method for operating a health management apparatus. The method comprises: a first acquisition step of acquiring medical examination results which are results of a medical examination of a pet; a second acquisition step of acquiring an optical image of the pet; and a screen output control step of controlling an output of a medical examination result display screen including the medical examination results and at least one of the optical image or a schema diagram created on the basis of the optical image.

According to the present disclosure, there is provided a program for operating a health management apparatus. The program causes a computer to function as: a first acquisition unit that acquires medical examination results which are results of a medical examination of a pet; a second acquisition unit that acquires an optical image of the pet; and a screen output control unit that controls an output of a medical examination result display screen including the medical examination results and at least one of the optical image or a schema diagram created on the basis of the optical image.

According to the technology of the present disclosure, it is possible to provide a health management apparatus that enables a user to smoothly understand the medical examination results of a pet, a method for operating the health management apparatus, and a program for operating the health management apparatus.

### BRIEF DESCRIPTION OF THE DRAWINGS

Exemplary embodiments according to the technique of the present disclosure will be described in detail based on the following figures, wherein:

FIG. 1 is a diagram illustrating a health management system;

FIG. 2 is a diagram illustrating the type of medical examination;

FIG. 3 is a diagram illustrating various kinds of information associated with medical examination results;

FIG. 4 is a diagram illustrating an aspect of an X-ray examination of a pet in an animal hospital;

FIG. 5 is a block diagram illustrating a computer forming each of a health management apparatus and an owner terminal;

FIG. 6 is a block diagram illustrating a processing unit of a CPU of the health management apparatus;

FIG. 7 is a diagram illustrating selection information;

FIG. 8 is a diagram illustrating an aspect in which a selection unit selects an important item;

FIG. 9 is a diagram illustrating an organ and bone DB;

FIG. 10 is a diagram illustrating an aspect in which a screen generation unit displays organs so as to be superimposed on an optical image;

FIG. 11 is a diagram illustrating an aspect in which positional information of an organ and bone which has not been registered in the organ and bone DB is created;

FIG. 12 is a diagram illustrating a process in which a screen generation unit displays a frame indicating an imaging region in an X-ray examination on an optical image;

FIG. 13 is a diagram illustrating a process in which the screen generation unit displays the frame indicating the imaging region in the X-ray examination on the optical image;

FIG. 14 is a block diagram illustrating a processing unit of a CPU of the owner terminal;

FIG. 15 is a diagram illustrating a medical examination result display screen;

FIG. 16 is a diagram illustrating the medical examination result display screen;

FIG. 17 is a diagram illustrating the medical examination result display screen;

FIG. 18 is a flowchart illustrating the procedure of a process of the health management apparatus;

FIG. 19 is a diagram illustrating an aspect in which a schema diagram is created on the basis of the optical image and is displayed on the medical examination result display screen;

FIG. 20 is a diagram illustrating an aspect in which a schema diagram is created on the basis of the optical image and the schema diagram and the optical image are displayed on the medical examination result display screen;

FIG. 21 is a diagram illustrating a state in which the frame is displayed so as to be superimposed on the schema diagram created on the basis of the optical image;

FIG. 22 is a diagram illustrating a third embodiment in which a display size of a frame is changed in operative association with the enlargement and reduction of a medical image. (A) of FIG. 22 illustrates the medical image before enlargement or after reduction and (B) of FIG. 22 illustrates the medical image before reduction or after enlargement;

FIG. 23 is a diagram illustrating a fourth embodiment in which a medical image is displayed in a frame and the transparency of a schema diagram in the frame is changed in response to an operation of the owner. (A) of FIG. 23 illustrates a case in which in which the transparency is 0% and (B) of FIG. 23 illustrates a case in which in which the transparency is 100%;

FIG. 24 is a diagram illustrating the display state of the schema diagram and the medical image in a case in which the transparency is 50%; and

FIG. 25 is a diagram illustrating a medical examination result display screen in a case in which a display of the owner terminal is aligned in the horizontal direction.

### DETAILED DESCRIPTION

### First Embodiment

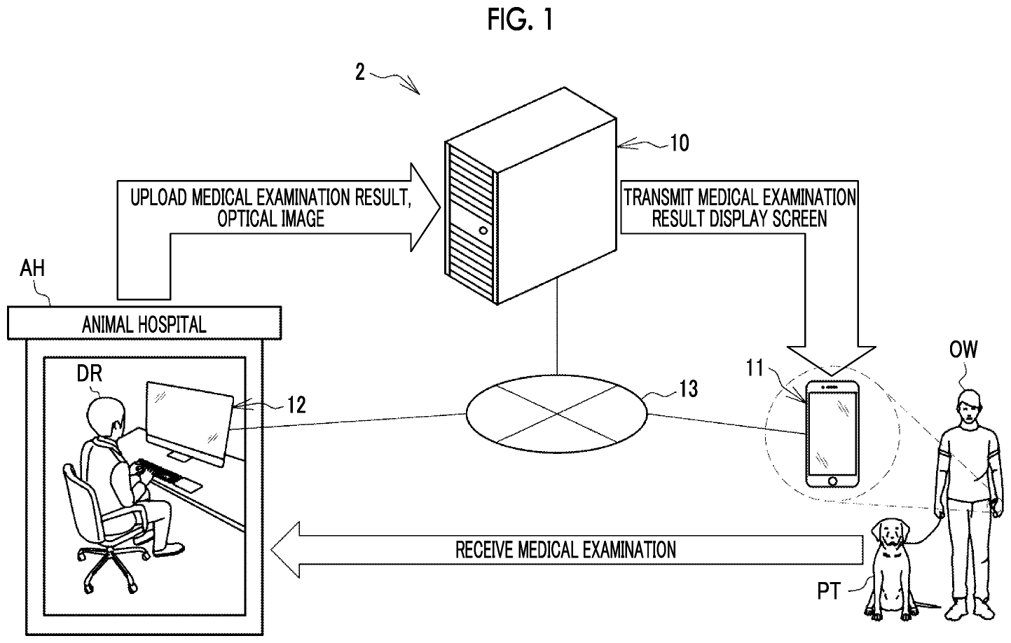

In FIG. 1, a health management system 2 is a system that manages a health state of a pet PT. The health management system 2 comprises a health management apparatus 10, an owner terminal 11 that is owned by an owner OW of the pet PT, and a hospital terminal 12 that is operated by, for example, a doctor DR in an animal hospital AH. The health management apparatus 10 is, for example, a server computer. The owner terminal 11 is, for example, a smart phone. The hospital terminal 12 is, for example, a desktop personal computer. In practice, there are a plurality of pets PT, owners OW, and animal hospitals AH. Therefore, in practice, there are a plurality of owner terminals 11 and a plurality of hospital terminals 12.

The health management apparatus 10, the owner terminal 11, and the hospital terminal 12 are connected through a network 13 so as to communicate with each other. The network 13 is a wide area network (WAN) such as the Internet or a public telecommunication network.

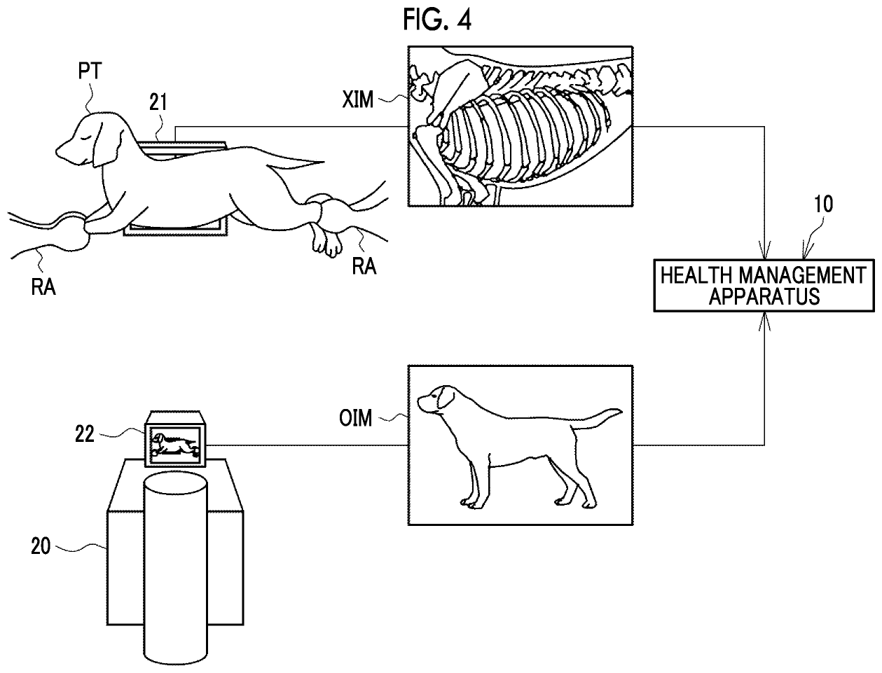

The owner OW visits the animal hospital AH with the pet PT such that the pet PT receives a medical examination at the animal hospital AH. The doctor DR operates the hospital terminal 12 to upload the medical examination results of the pet PT to the health management apparatus 10. In addition, the doctor DR uploads an optical image OIM (see, for example, FIG. 4) of the pet PT to the health management apparatus 10 in addition to the medical examination results.

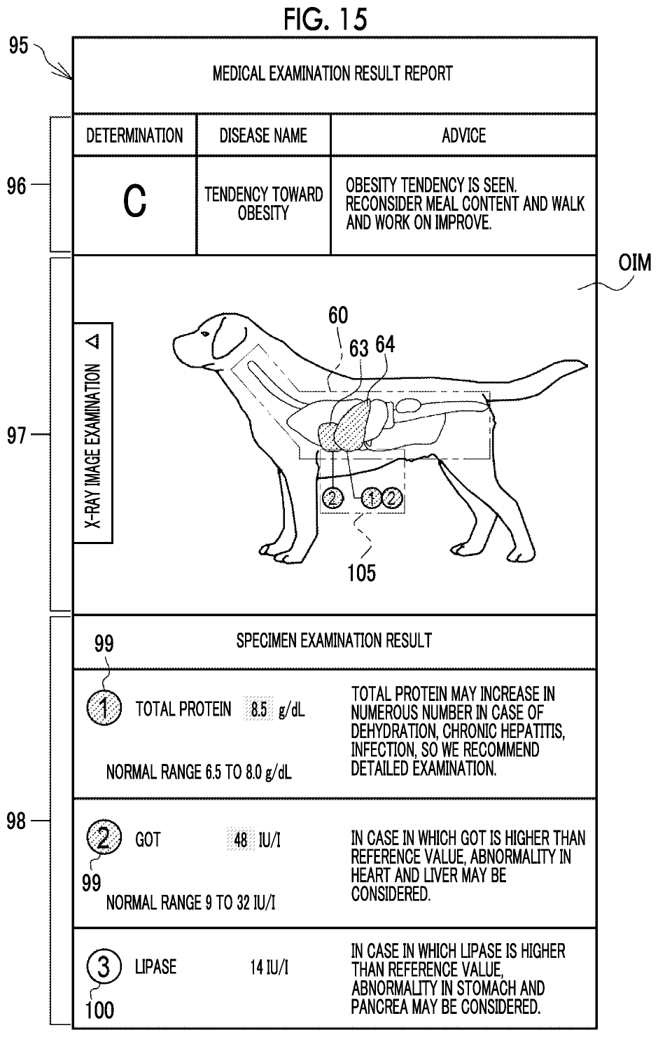

The health management apparatus 10 transmits an upload notification indicating that the medical examination results and the optical image OIM have been uploaded from the hospital terminal 12 to the owner terminal 11. In a case in which the upload notification is received, the owner OW operates the owner terminal 11 to transmit a transmission request to the health management apparatus 10. The health management apparatus 10 receives the transmission request from the owner terminal 11 and transmits a medical examination result display screen 95 (see, for example, FIG. 15) including the medical examination results and the optical image OIM to the owner terminal 11. The owner OW is an example of a “user” according to the technology of the present disclosure.

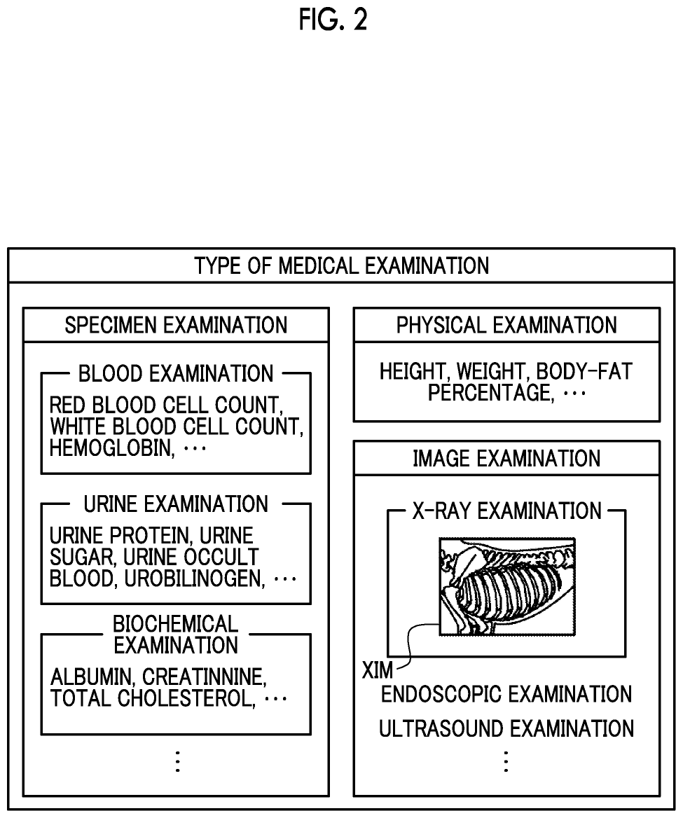

In FIG. 2, the types of medical examinations are mainly divided into a specimen examination, a physical examination, and an image examination. Examples of the specimen examination include a blood examination, a urine examination, and a biochemical examination. The blood examination includes items, such as a red blood cell count, a white blood cell count, and hemoglobin. The urine examination includes items, such as urine protein, urine sugar, urine occult blood, and urobilinogen. The biochemical examination includes items, such as albumin, creatinine, and total cholesterol. The physical examination includes items, such as height, weight, and body-fat percentage. The results of the specimen examination and the physical examination are the numerical values of the above-mentioned items. Examples of the image examination include an X-ray examination, an endoscopic examination, and an ultrasound examination. The result of the image examination is a medical image whose example is an X-ray image XIM in the X-ray examination.

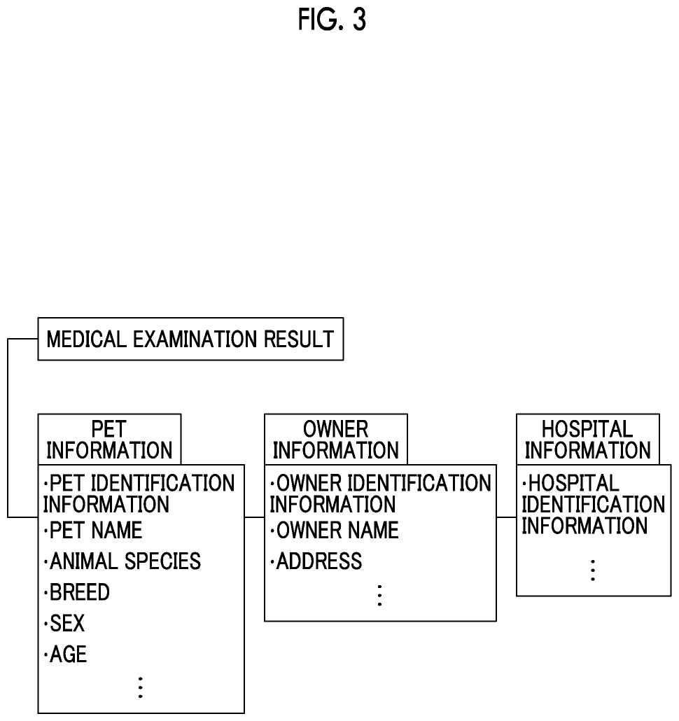

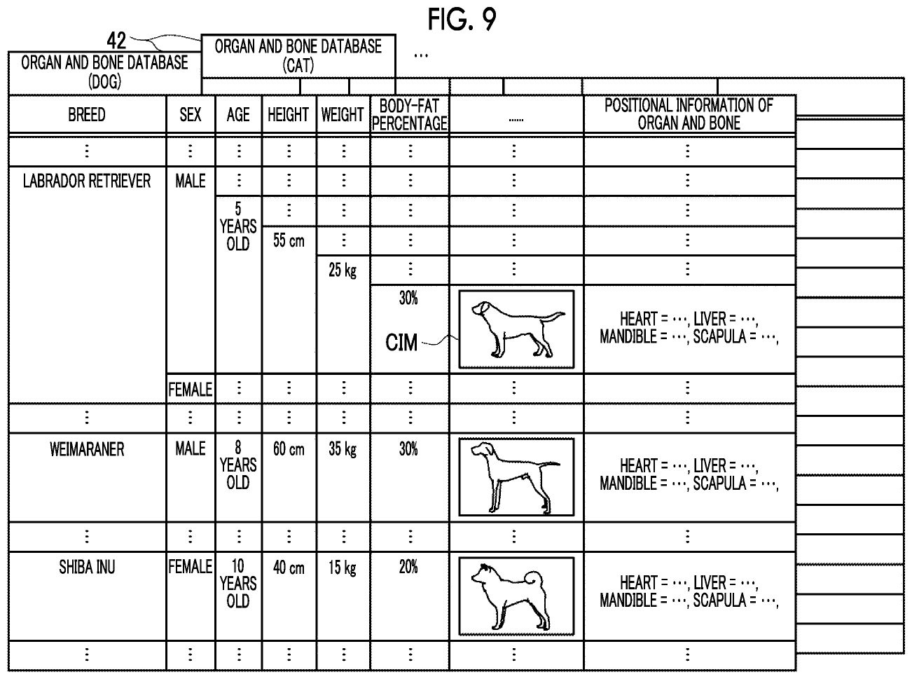

Pet information, owner information, and hospital information illustrated in FIG. 3 are associated with the medical examination results. The pet information includes, for example, pet identification information, a pet name, an animal species, a breed, sex, and age. The pet identification information is identification data (ID) including a symbol and/or a number. Examples of the animal species include a dog and a cat. In a case in which the pet PT is a dog, the breed is, for example, Labrador Retriever or Shiba Inu (see FIG. 9). The owner information includes, for example, owner identification information, an owner name, and an address. The hospital information includes, for example, hospital identification information. The pet information, the owner information, and the hospital information are uploaded from the hospital terminal 12 to the health management apparatus 10 together with the medical examination results.

In FIG. 4 illustrating an aspect of the X-ray examination of the pet PT in the animal hospital AH, an X-ray source 20 and an X-ray detector 21 are used in the X-ray examination. The X-ray source 20 emits X-rays to the pet PT. The X-ray detector 21 detects the X-rays which have been emitted from the X-ray source 20 and then transmitted through the pet PT and outputs an X-ray image XIM. The X-ray detector 21 is, for example, a flat panel detector (FPD) having an imaging surface in which pixels converting X-rays into an electric signal are two-dimensionally arranged. The X-ray detector 21 may be an indirect conversion type which includes a scintillator converting X-rays into visible light and converts the visible light emitted from the scintillator into an electric signal or a direct conversion type which directly converts X-rays into an electric signal.

In the X-ray examination, for example, the pet PT lies on its side on a bed (not illustrated) and the limbs are held by a radiology technician RA who performs the X-ray examination. Then, the X-ray detector 21 is inserted under the pet PT. The X-ray examination may be performed with the pet PT lying on its face or lying on its back. In addition, in some cases, the front legs and waist of the pet PT may be held by the radiology technician RA.

A digital camera 22 for capturing the optical image OIM of the pet PT is attached to the X-ray source 20. The digital camera 22 is operated by the radiology technician RA. An imaging guide is displayed on a viewfinder of the digital camera 22. The imaging guide is, specifically, a side standing contour image of the pet PT with a recommended angle of view (position and size). The imaging guide is prepared for each animal species, breed, sex, and age. The radiology technician RA performs an operation of displaying an imaging guide suitable for the pet PT on the viewfinder of the digital camera 22 and takes an image of the side standing position of the pet PT according to the imaging guide in a place where there is nothing in the background. Then, the optical image OIM of the pet PT with an angle of view that is nearly the same as the recommended angle of view is captured.

A portable X-ray source, such as a product name “CALNEO Xair” (manufactured by FUJIFILM Corporation) may be used as the X-ray source 20. In a case in which the portable X-ray source is used, the pet PT in the standing position may be irradiated with X-rays to capture the X-ray image XIM as in the case in which the optical image OIM is captured, unlike the case in which the PET PT lies on its side on the bed and the limbs of the pet PT are held by the radiology technician RA as illustrated in FIG. 4. In a case in which the pet PT lies on its side on the bed, there is a concern that the pet PT will be exposed to radiation. Therefore, the radiology technician RA needs to hold, for example, the limbs of the pet PT. In contrast, in a case in which the portable X-ray source is used, the radiology technician RA does not need to hold, for example, the limbs of the pet PT. Therefore, it is possible to capture the X-ray image XIM of the pet PT in a more natural standing position. Further, it is possible to continuously perform the capture of the X-ray image XIM and the capture of the optical image OIM.

The X-ray image XIM output from the X-ray detector 21 and the optical image OIM captured by the digital camera 22 are uploaded to the health management apparatus 10. In addition, the optical image OIM of the pet PT may not be captured by the digital camera 22 attached to the X-ray source 20, but may be captured by, for example, a smart phone of the radiology technician RA. Then, the captured optical image OIM may be uploaded to the health management apparatus 10.

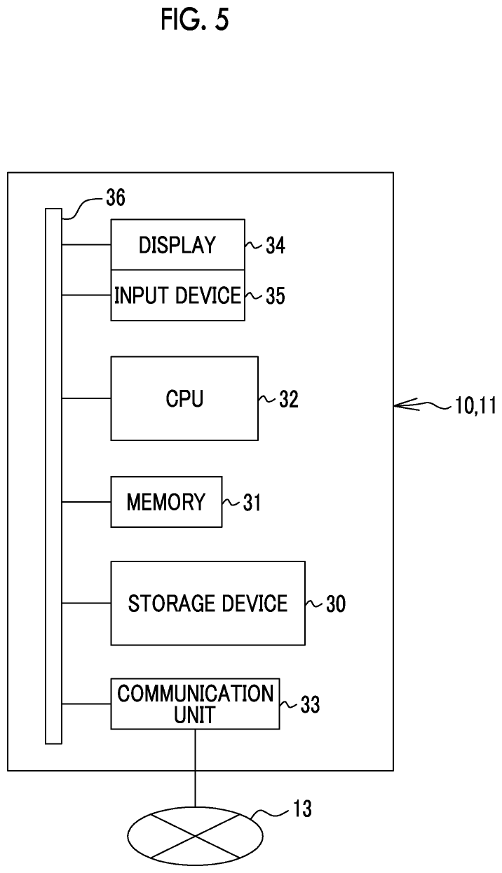

In FIG. 5, computers forming the health management apparatus 10 and the owner terminal 11 basically have the same configuration and comprise a storage device 30, a memory 31, a central processing unit (CPU) 32, a communication unit 33, a display 34, and an input device 35. These components are connected to each other through a bus line 36.

The storage device 30 is a hard disk drive that is provided in the computer forming each of the health management apparatus 10 and the owner terminal 11 or is connected to the computer through a cable or a network. Alternatively, the storage device 30 is a disk array in which a plurality of hard disk drives are connected in series to each other. The storage device 30 stores, for example, a control program, such as an operating system, or various application programs (hereinafter, abbreviated to APs) and various types of data associated with the programs.

The memory 31 is a work memory used by the CPU 32 to perform processes. The CPU 32 loads the program stored in the storage device 30 to the memory 31 and performs the process based on the program to control the overall operation of each unit of the computer.

The communication unit 33 is a network interface that controls the transmission of various kinds of information through the network 13. The display 34 displays various screens. The various screens include operation functions by a graphical user interface (GUI). The computers forming the health management apparatus 10 and the owner terminal 11 receive operation commands input from the input device 35 through various screens. The input device 35 is, for example, a keyboard, a mouse, or a touch panel.

In the following description, a suffix “A” is attached to each component of the computer forming the health management apparatus 10 and a suffix “B” is attached to each component of the computer forming the owner terminal 11 to distinguish the components.

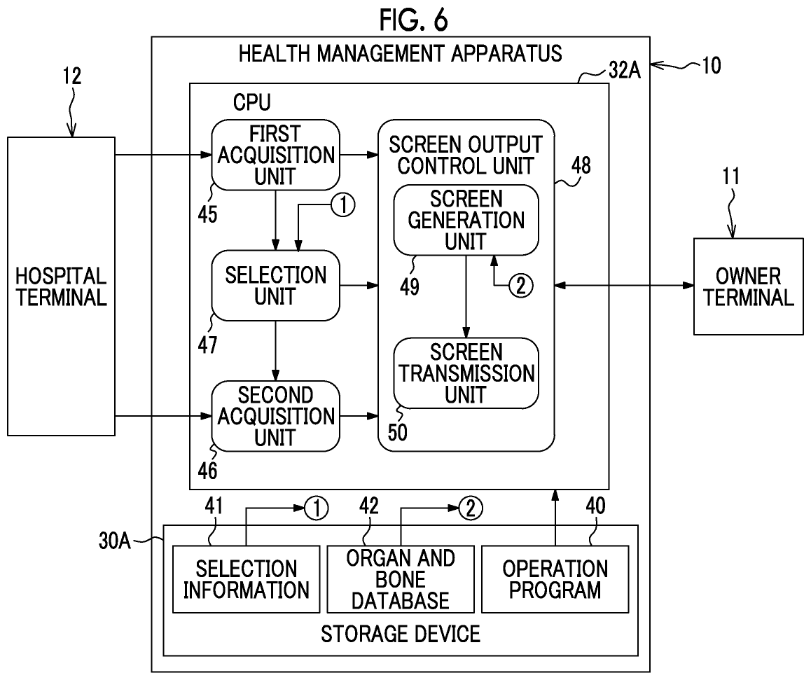

In FIG. 6, a storage device 30A of the health management apparatus 10 stores an operation program 40. The storage device 30A stores selection information 41 and an organ and bone database (hereinafter, abbreviated to DB) 42 in addition to the operation program 40.

The operation program 40 is an example of a “program for operating a health management apparatus” according to the technology of the present disclosure. In a case in which the operation program 40 is run, the CPU 32A of the health management apparatus 10 functions as a first acquisition unit 45, a second acquisition unit 46, a selection unit 47, and a screen output control unit 48 in cooperation with, for example, the memory 31. The screen output control unit 48 includes a screen generation unit 49 and a screen transmission unit 50. The concept of the “screen output control unit 48” described below includes the screen generation unit 49 and the screen transmission unit 50.

The first acquisition unit 45 acquires the medical examination results of the pet PT from the hospital terminal 12. The first acquisition unit 45 outputs the acquired medical examination results to the selection unit 47 and the screen output control unit 48. The second acquisition unit 46 acquires the optical image OIM of the pet PT from the hospital terminal 12. The second acquisition unit 46 outputs the acquired optical image OIM to the screen output control unit 48.

The selection unit 47 selects an important item from the items of the medical examination on the basis of the selection information 41. The selection unit 47 outputs the selected important item to the screen output control unit 48.

The screen output control unit 48 receives a request to transmit various screens including the medical examination result display screen 95 from the owner terminal 11. In a case in which the transmission request is received, the screen output control unit 48 controls the output of various screens to the owner terminal 11. Specifically, the screen output control unit 48 outputs various screens in the form of screen data for web transmission created by a markup language, such as Extensible Markup Language (XML). Then, it is possible to browse various screens on a web browser with the owner terminal 11. Instead of XML, other data description languages including Javascript (registered trademark) Object Notation (JSON) may be used instead of XML.

The screen generation unit 49 generates screen data for various screens and outputs the generated screen data to the screen transmission unit 50. The screen transmission unit 50 transmits the screen data from the screen generation unit 49 to the owner terminal 11 which is the source of the request to transmit various screens.

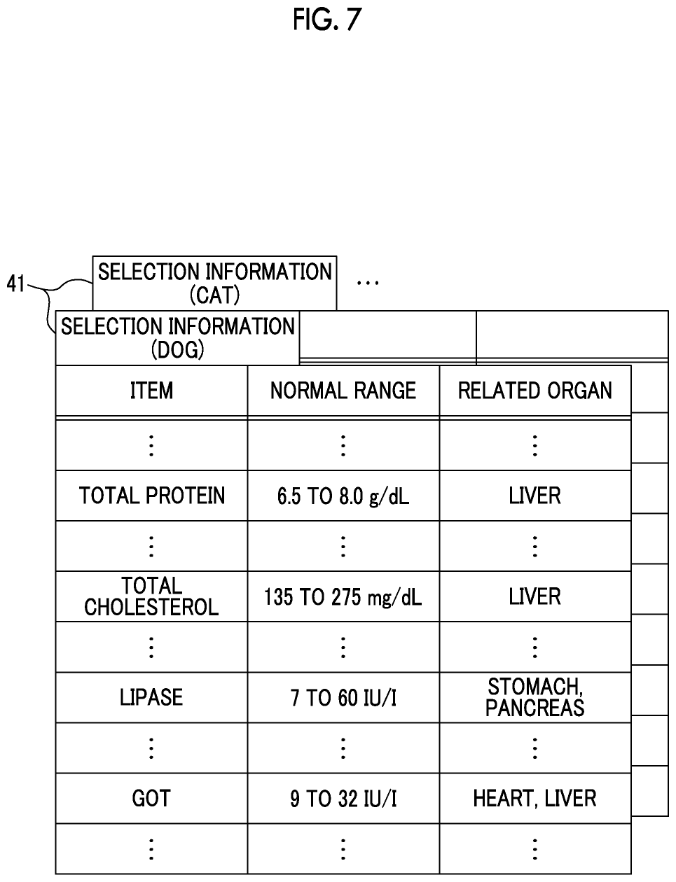

In FIG. 7, the selection information 41 is information related to the normal ranges of the numerical values of the items of the specimen examination. Specifically, the selection information 41 is information in which the normal ranges of the numerical values of each item of the specimen examination and the organs related to the items are registered. There are a plurality of kinds of selection information 41 according to the animal species of the pet PT such as a dog and a cat.

FIG. 7 illustrates the selection information 41 of a dog. In the example illustrated in FIG. 7, the normal range of total cholesterol is “135 mg/dL to 275 mg/dL”, the related organ is “the liver”, the normal range of glutamic oxaloacetic transaminase (GOT) is “9 IU/I to 32 IU/I”, and the related organs are “the heart and the liver”.

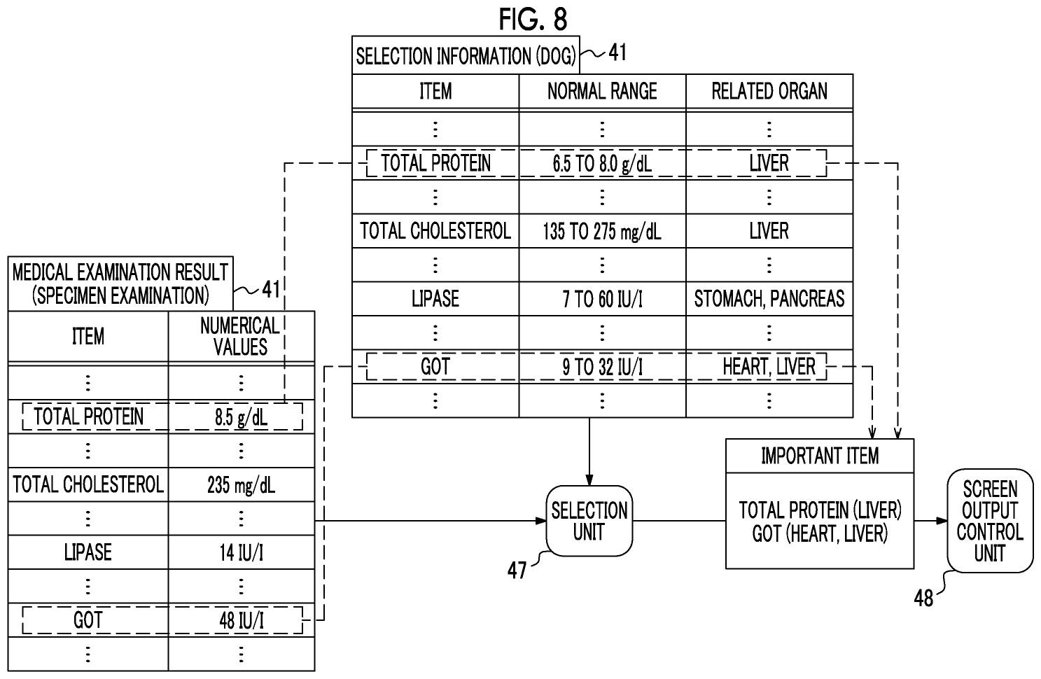

As illustrated in FIG. 8, the selection unit 47 compares the numerical value of each item of the medical examination results from the first acquisition unit 45 with the normal range of the selection information 41. Then, the selection unit 47 selects the item whose numerical value is out of the normal range as the important item. In other words, the item whose numerical value is out of the normal range is an item with an abnormal numerical value. The item with an abnormal numerical value is an item to which the owner OW needs to pay particular attention in checking the health status of the pet PT. The selection unit 47 outputs the selected important item and the organ related to the important item to the screen output control unit 48.

FIG. 8 illustrates a case in which the numerical value of total protein in the medical examination results is 8.5 g/dL and is out of the normal range of 6.5 g/dL to 8.0 g/dL and the numerical value of GOT in the medical examination results is 48 IU/I and is out of the normal range of 9 IU/I to 32 IU/I. In this case, the selection unit 47 selects total protein and GOT as the important items and outputs the “heart” and the “liver” related to the total protein item and the GOT item to the screen output control unit 48 together with the total protein item and the GOT item.

In FIG. 9, in the organ and bone DB 42, the side standing contour image CIM of the pet PT and the positional information of the organ and bone in the contour image CIM are registered for each of the breed, sex, age, height, weight, and body-fat percentage of the pet PT. For example, the age is in increments of 1 year in the range of 0 to 15 years old. For example, the height varies depending on the breed and is in increments of 1 cm in the range of 50 cm to 60 cm. For example, the weight also depends on the breed and is in increments of 1 kg in the range of 20 kg to 30 kg. For example, the body-fat percentage is in increments of 5% in the range of 10% to 30%.

For example, the contour image CIM is generated by extracting the contour of the pet PT from a plurality of optical images OIM captured in the past and averaging the contours. Examples of the contour image CIM include an image that is displayed as the imaging guide on the viewfinder of the digital camera 22. Specifically, the positional information of the organ is a set of the coordinates of a plurality of pixels corresponding to the contour of each organ, such as the heart or the liver, in the contour image CIM. The positional information of the bone is a set of the coordinates of a plurality of pixels corresponding to the contour of a specific bone, such as the mandible or the scapula included in the X-ray image XIM, in the contour image CIM. The positional information of the organ and bone is, for example, information derived by averaging the positions of the organ and bone included in a plurality of X-ray images XIM captured in the past. There are a plurality of types of organ and bone DBs 42 according to the animal species of the pet PT, such as a dog and a cat, similarly to the selection information 41.

FIG. 9 illustrates the organ and bone DB 42 of a dog. FIG. 9 illustrates the contour image CIM and the positional information of the organ and bone of a dog that is a breed of “Labrador Retriever”, is a “male”, is “5 years old”, and has a height of “55 cm”, a weight of “25 kg”, and a body-fat percentage of “30%”. In addition, FIG. 9 illustrates the contour image CIM and the positional information of the organ and bone of a dog that is a breed of “Weimaraner”, is a “male”, is “8 years old”, and has a height of “60 cm”, a weight of “35 kg”, and a body-fat percentage of “15%”. Further, FIG. 9 illustrates the contour image CIM and the positional information of the organ and bone of a dog that is a breed of “Shiba Inu”, is a “female”, is “10 years old”, and has a height of “40 cm”, a weight of “15 kg”, and a body-fat percentage of “20%”.

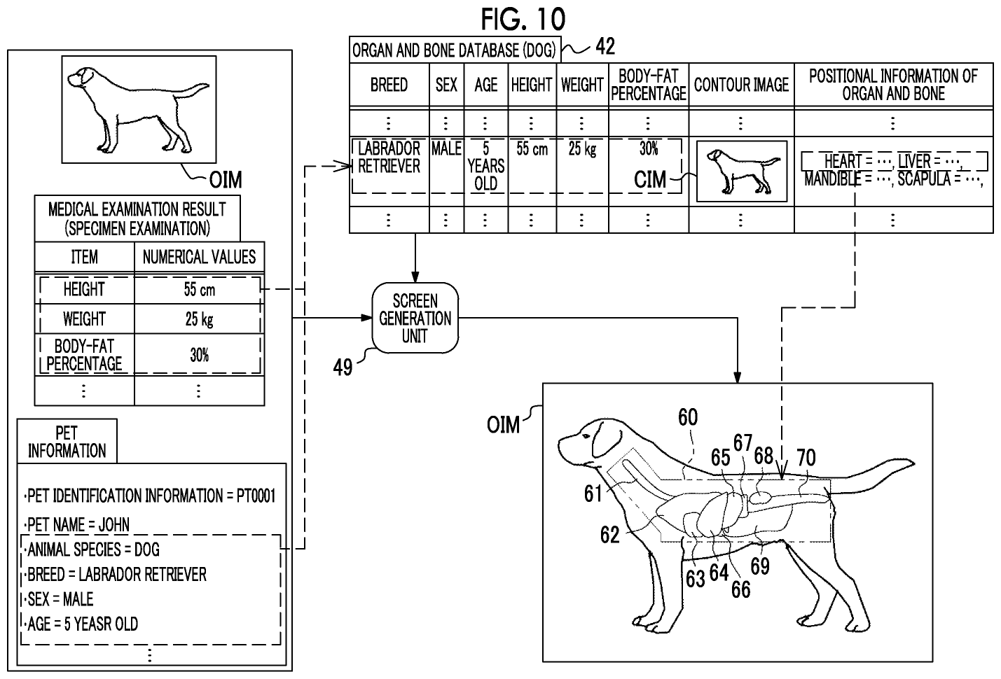

In FIG. 10, the screen generation unit 49 displays organs 60 so as to be superimposed on the optical image OIM on the basis of the organ and bone DB 42. Specifically, the screen generation unit 49 searches for the positional information of the organs corresponding to the medical examination results (numerical values) of the height, weight, and body-fat percentage in the physical examination and the animal species, breed, sex, and age in the pet information from the organ and bone DB 42, using these items as keys. Then, the screen generation unit 49 draws the organs 60 on the optical image OIM on the basis of the searched positional information of the organs. The organs 60 include the gullet 61, the lung 62, the heart 63, the liver 64, the stomach 65, the pancreas 66, the spleen 67, the kidney 68, the small intestine 69, and the large intestine 70.

FIG. 10 illustrates a case in which the pet PT is an animal species of “dog”, is a breed of “Labrador Retriever”, is a “male”, is “5 years old”, and has a height of “55 cm”, a weight of “25 kg”, and a body-fat percentage of “30%”. In this case, the screen generation unit 49 searches for the positional information of the organs corresponding to a dog that is a breed of “Labrador Retriever”, is a “male”, is “5 years old”, and has a height of “55 cm”, a weight of “25 kg”, and a body-fat percentage of “30%” from the organ and bone DB 42 of the dog. Then, the screen generation unit 49 draws the organs 60 on the optical image OIM on the basis of the searched positional information.

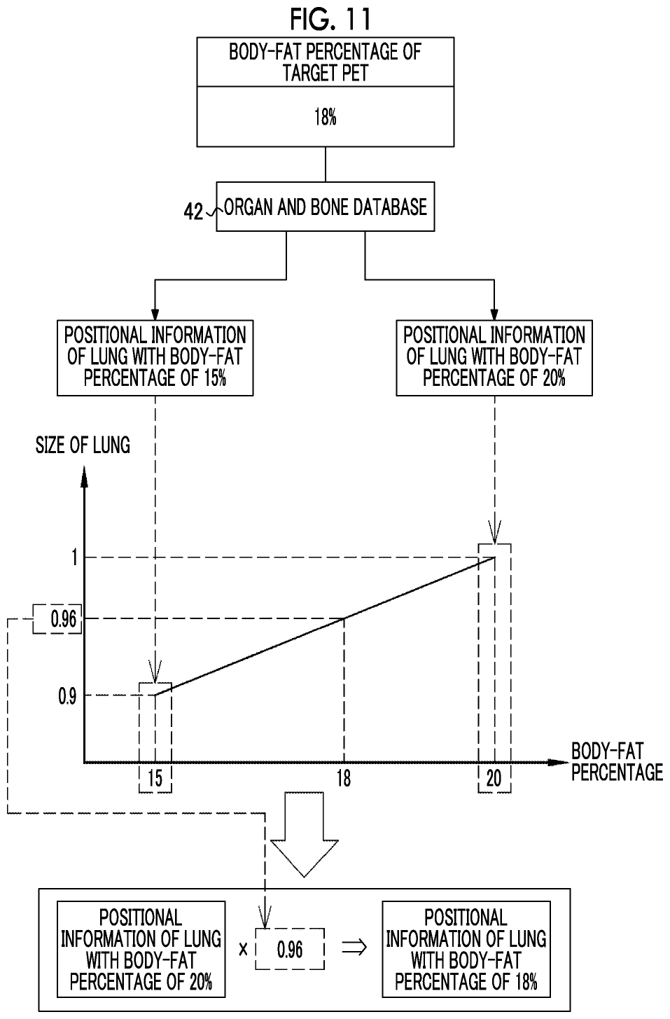

In the above-mentioned example, the body-fat percentage of the organ and bone DB 42 is, for example, in increments of 5%. In this case, for example, positional information including a body-fat percentage of 18% between the body-fat percentages of the registered positional information of the organ and bone has not been registered. As such, in a case in which positional information has not been registered, as illustrated in FIG. 11, the screen generation unit 49 creates the positional information of the organ and bone which has not been registered from the registered positional information of the organ and bone by analogy.

As illustrated in FIG. 11, in the creation of the positional information which has not been registered in the organ and bone DB 42, first, the screen generation unit 49 searches for the positional information of two body-fat percentages on the upper and lower sides of the body-fat percentage of the target pet PT from the organ and bone DB 42. Then, the screen generation unit 49 calculates the size of the organ and bone on the basis of the positional information of the two body-fat percentages. The size of the organ and bone is synonymous with the area of the organ and bone.

Then, a relational expression between the size of the organ and bone and the body-fat percentage is derived from the calculated size of the organ and bone. The relational expression is a linear function represented by the following Expression (1) in a case in which the size of the organ and bone is S, the body-fat percentage is BFP, and coefficients are α and β:

S=αBFP+β (1).

In a case in which the relational expression is derived, the size of the organ and bone calculated on the basis of the positional information of the higher of the two body-fat percentages on the upper and lower sides of the body-fat percentage of the target pet PT is normalized to 1.

Then, the body-fat percentage of the target pet PT is substituted into the derived relational expression to calculate the size of the organ and bone of the target pet PT. Then, the size of the organ and bone with the higher of the body-fat percentages is multiplied by the calculated size of the organ and bone to create the positional information which has not been registered in the organ and bone DB 42.

FIG. 11 illustrates a case in which the body-fat percentage of the target pet PT is 18% and the positional information of the lung is created. In this case, as the positional information of two body-fat percentages, the positional information of the lung with a body-fat percentage of 15% and the positional information of the lung with a body-fat percentage of 20% are searched from the organ and bone DB 42. The positional information of the lung with a body-fat percentage of 20% corresponds to “the positional information of the higher of two body-fat percentages”.

A relational expression is derived on the basis of the positional information of the lung with a body-fat percentage of 15% and the positional information of the lung with a body-fat percentage of 20% and the size of the lung of the target pet PT with a body-fat percentage of 18% is calculated. As a result, the size of the lung is 0.96. Therefore, the screen generation unit 49 multiplies the size of the lung with a body-fat percentage of 20% by 0.96 to create the positional information of the lung with a body-fat percentage of 18%.

FIG. 11 illustrates a method that creates positional information which has not been registered in the organ and bone DB 42 from the relational expression which is a linear function derived on the basis of the positional information of two body-fat percentages on the upper and lower sides of the body-fat percentage of the target pet PT. This method is called a linear interpolation method. In a case in which the body-fat percentage of the target pet PT is out of the range of the body-fat percentage in the organ and bone DB 42, linear extrapolation may be performed instead of the linear interpolation.

In FIG. 11, the size of the organ and bone is calculated from the positional information registered in the organ and bone DB 42 and the positional information which has not been registered in the organ and bone DB 42 is created on the basis of the calculated size of the organ and bone. However, the present disclosure is not limited thereto. The position of the organ and bone may be calculated from the positional information registered in the organ and bone DB 42 and the positional information which has not been registered in the organ and bone DB 42 may be created on the basis of the calculated position of the organ and bone. However, the position of the organ and bone does not vary significantly depending on the body-fat percentage, as compared to the size of the organ and bone. Therefore, the position of the organ and bone is the same regardless of the body-fat percentage and positional information based on the position of the organ and bone may not be created.

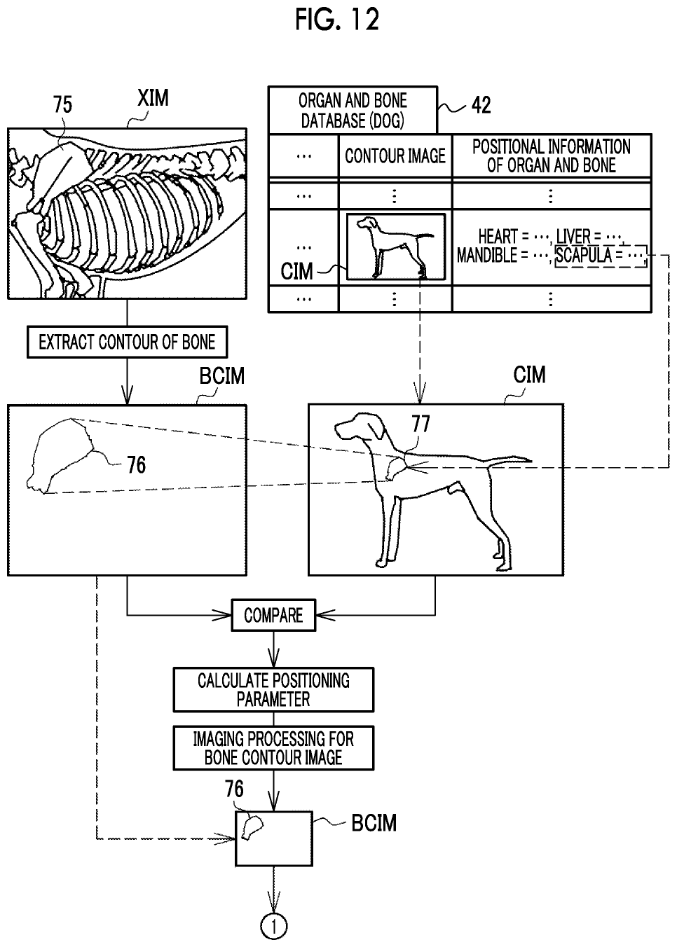

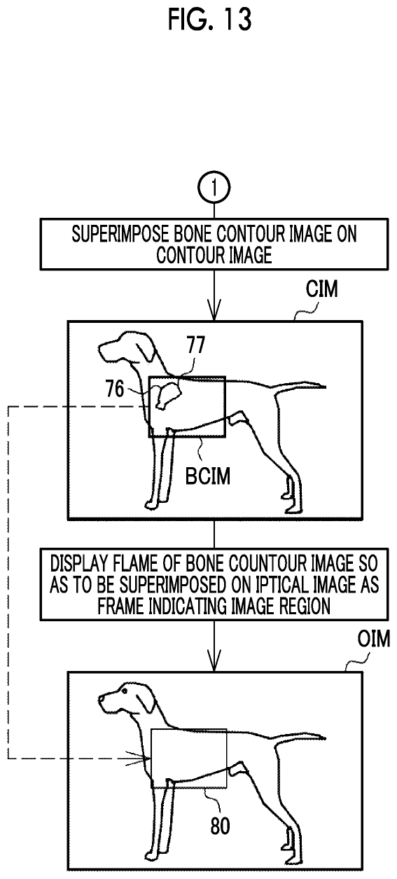

As illustrated in FIGS. 12 and 13, the screen generation unit 49 displays a frame 80 indicating an imaging region in the X-ray examination so as to be superimposed on a part of the body of the pet PT drawn in the optical image OIM. Specifically, first, the screen generation unit 49 generates a bone contour image BCIM obtained by extracting a contour 76 of the specific bone 75 from the X-ray image XIM. In addition, the screen generation unit 49 searches for a contour image CIM corresponding to the target pet PT and the positional information of the same bone as the specific bone 75 from the organ and bone DB 42.

The screen generation unit 49 compares the contour 76 of the bone 75 in the bone contour image BCIM with a contour 77 of the bone based on the positional information searched from the organ and bone DB 42. As a result, the screen generation unit 49 calculates a positioning parameter for matching the contour 76 with the contour 77. Examples of the positioning parameter include the enlargement ratio, reduction ratio, and rotation amount of the bone contour image BCIM.

The screen generation unit 49 performs imaging processing, such as an enlargement process, a reduction process, or a rotation process, for the bone contour image BCIM on the basis of the calculated positioning parameter. Then, the screen generation unit 49 superimposes the bone contour image BCIM on the contour image CIM such that the contour 76 is matched with the contour 77. The screen generation unit 49 displays a frame of the bone contour image BCIM superimposed on the contour image CIM as the frame 80 indicating the imaging region in the X-ray examination so as to be superimposed on the optical image OIM.

In FIGS. 12 and 13, the scapula is illustrated as the specific bone 75. However, the specific bone 75 is not limited to the scapula and may be any bone included in the X-ray image XIM, such as the mandible, the humerus, the femur, or the pelvis.

A method for displaying the frame 80 indicating the imaging region in the X-ray examination so as to be superimposed on the optical image OIM is not limited to the method illustrated in FIGS. 12 and 13. For example, the enlargement ratio and reduction ratio of the bone contour image BCIM may be calculated on the basis of the distance between two specific bones included in the X-ray image XIM. Alternatively, the display position of the frame 80 in the optical image OIM may be defined on the basis of the distance between the specific bone 75 and the edge of the X-ray image XIM.

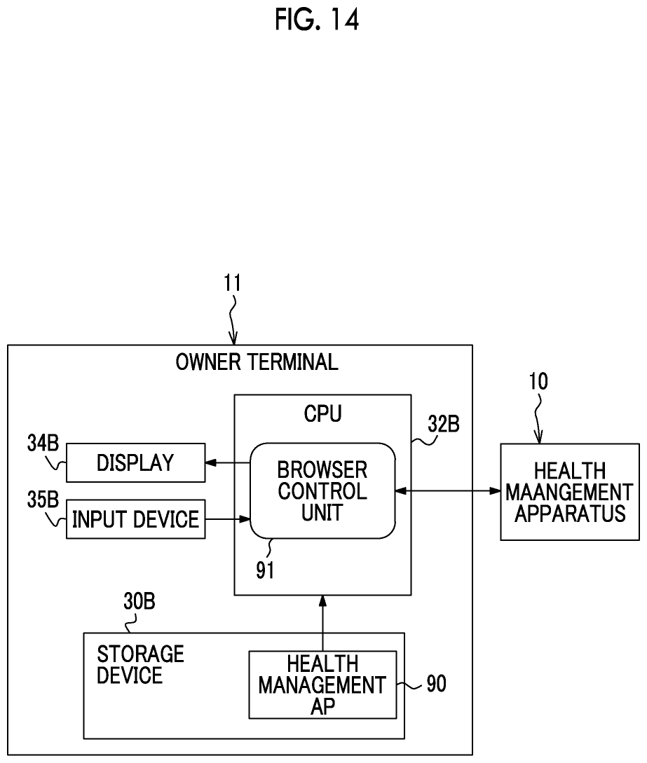

In FIG. 14, a health management AP 90 is stored in a storage device 30B of the owner terminal 11. In a case in which the health management AP 90 is executed to start up a web browser, a CPU 32B of the owner terminal 11 functions as a browser control unit 91 in cooperation with, for example, the memory 31. The browser control unit 91 controls the operation of the web browser. The browser control unit 91 receives screen data of various screens from the health management apparatus 10. The browser control unit 91 reproduces various screens displayed on the web browser on the basis of the screen data and displays the screens on a display 34B.

In addition, the browser control unit 91 receives various operation commands input from an input device 35B by the owner OW through various screens. The operation commands include a command to access the health management apparatus 10 and a command to transmit various screens. The browser control unit 91 issues a request corresponding to the operation command to the health management apparatus 10. For example, the browser control unit 91 issues a request to transmit various screens to the health management apparatus 10 in response to the command to transmit various screens. Since the owner terminal 11 is a smart phone, the input device 35B is a touch panel.

In FIGS. 15 to 17, a medical examination result display screen 95 displayed on the display 34B of the owner terminal 11 is mainly divided into a summary display region 96, an optical image display region 97, and a medical examination result display region 98. The determination result of the level of the health state of the pet PT obtained by comprehensively considering the medical examination results, a disease name derived from the medical examination results, and advice for the owner OW are displayed in the summary display region 96. The optical image OIM is displayed in the optical image display region 97. The medical examination results are displayed in the medical examination result display region 98. That is, the medical examination result display screen 95 includes the optical image OIM and the medical examination results.

In FIG. 15, the organs 60 are displayed so as to be superimposed on the optical image OIM. The numerical value and normal range of each item of the specimen examination and simple comments are displayed in the medical examination result display region 98. The screen generation unit 49 displays the important item selected by the selection unit 47 in a display format that is different from the display format of other items on the medical examination result display screen 95. Specifically, the important item is displayed at a higher position than other items. In addition, the number 99 assigned to the important item is, for example, a white letter on a red background as illustrated by hatching. On the other hand, the number 100 assigned to another item is, for example, a white letter on a green background. In addition, for example, while the numerical value of the important item is displayed in red as illustrated by hatching, the numerical value of another item is displayed in green.

Further, the screen generation unit 49 displays an annotation 105 indicating the organ related to the important item on the optical image OIM. The annotation 105 includes a line indicating the organ related to the important item at one end and the number 99 assigned to the important item disposed at the other end of the line. As illustrated by hatching, for example, while the organ related to the important item is painted in red, the organ that is not related to the important item is painted in gray.

In addition, as a method for displaying the important item in a display format different from the display format of other items, the following are considered. For example, only the important item is displayed and other items are not displayed. Alternatively, the important item is displayed in boldface and other items are displayed in lightface. Furthermore, the important item may be displayed with letters having a relatively large size and other items may be displayed with letters having a smaller size than the letters of the important item. Of course, the above-mentioned methods may be combined. Further, the line indicating the organ related to the important item may be removed and the annotation 105 may be formed by only the number 99. In this case, the number 99 is displayed so as to be superimposed on the organ related to the important item.

FIG. 15 illustrates a case in which the total protein and GOT illustrated in FIG. 8 are selected as the important items. In this case, since the organs related to the important items are the “heart” and the “liver”, the annotation 105 is displayed in the heart 63 and the liver 64. In addition, the heart 63 and the liver 64 are painted in red. In a case in which there are a plurality of organs related to the important items as in this example, the organs may be painted in different colors.

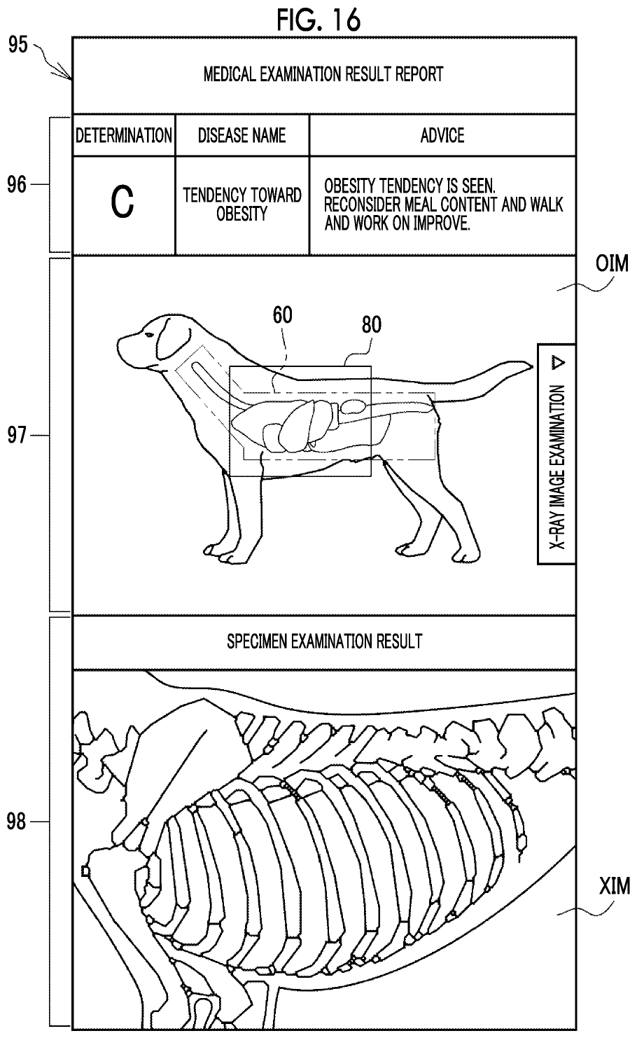

FIG. 16, the organs 60 are displayed so as to be superimposed on the optical image OIM as in the case illustrated in FIG. 15 and the frame 80 indicating the imaging region in the X-ray examination is displayed. The X-ray image XIM is displayed in the medical examination result display region 98. As such, the screen generation unit 49 displays the X-ray image XIM in a region different from the region in which the frame 80 is displayed. In addition, the frame 80 indicating an imaging region in an examination other than the X-ray examination, for example, an endoscopic examination or an ultrasound examination may be displayed and an endoscopic image or an ultrasound image may be displayed in the medical examination result display region 98.

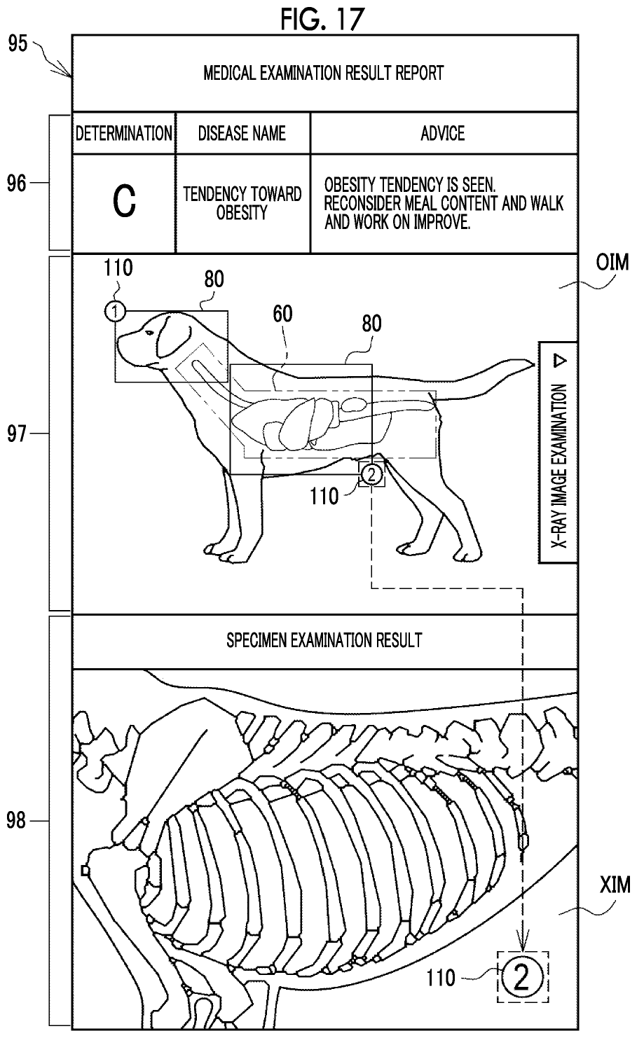

FIG. 17 illustrates a medical examination result display screen 95 in a case in which the image of a plurality of regions (two regions in FIG. 17) is captured in the X-ray examination. In this case, a plurality of frames 80 indicating the imaging regions in the X-ray examination are displayed on the optical image OIM. The plurality of frames 80 are configured so as to be individually selected by the owner OW. The X-ray image XIM corresponding to the frame 80 selected by the owner OW is displayed in the medical examination result display region 98.

A number 110 is assigned to the plurality of frames 80. The number 110 is also assigned to the X-ray image XIM in the medical examination result display region 98. The number 110 indicates which of the plurality of frames 80 is the frame 80 indicating the imaging region of the X-ray image XIM currently displayed in the medical examination result display region 98.

In addition, the following configuration may be used: for example, the doctor DR sets viewing priorities to a plurality of X-ray images XIM and the frame 80 of an X-ray image XIM having a high priority is displayed more conspicuously than the frame 80 of an X-ray image XIM having a low priority. For example, the frame 80 of the X-ray image XIM having a high priority is represented by a thick line and the frame 80 of the X-ray image XIM having a low priority is represented by a thin line. In this case, the X-ray image XIM initially displayed in the medical examination result display region 98 is an X-ray image XIM having a high priority.

The display of the medical examination result display screen 95 illustrated in FIG. 15 and the medical examination result display screen 95 illustrated in FIG. 16 or FIG. 17 can be switched by a swipe operation for the touch panel which is the input device 35B. In addition, a screen showing all of the medical examination results in a list format may be displayed in addition to the medical examination result display screen 95 illustrated in FIG. 15 and the medical examination result display screen 95 illustrated in FIG. 16 or FIG. 17. Further, a screen showing a treatment policy of the disease displayed on the summary display region 96 may be displayed. The treatment policy includes, for example, a recommended walk distance and time and a recommended meal amount. Furthermore, a map screen showing a recommended walk course or a recommended pet food purchase screen may be displayed in response to the operation of the owner OW.

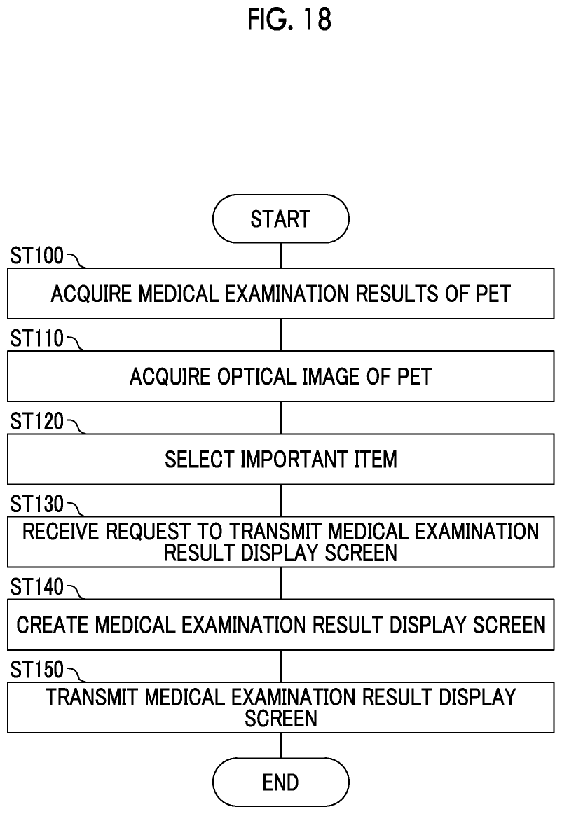

Next, the operation of the above-mentioned configuration will be described with reference to a flowchart illustrated in FIG. 18. First, in a case in which the operation program 40 is run, as illustrated in FIG. 6, the CPU 32A of the health management apparatus 10 functions as the first acquisition unit 45, the second acquisition unit 46, the selection unit 47, and the screen output control unit 48 (the screen generation unit 49 and the screen transmission unit 50).

The medical examination results of the pet PT and the optical image OIM of the pet PT are uploaded from the hospital terminal 12 of the animal hospital AH to the health management apparatus 10. The medical examination results of the pet PT are acquired by the first acquisition unit 45 (Step ST100). Step ST100 is an example of a “first acquisition step” according to the technology of the present disclosure. The medical examination results of the pet PT are output from the first acquisition unit 45 to the selection unit 47 and the screen output control unit 48.

The optical image OIM of the pet PT is acquired by the second acquisition unit 46 (Step ST110). Step ST110 is an example of a “second acquisition step” according to the technology of the present disclosure. The optical image OIM of the pet PT is output from the second acquisition unit 46 to the screen output control unit 48.

The selection unit 47 selects an important item on the basis of the selection information 41 stored in the storage device 30A in advance as illustrated in FIG. 8 (Step ST120). The important item is output from the selection unit 47 to the screen output control unit 48.

The screen output control unit 48 receives a request to transmit the medical examination result display screen 95 from the owner terminal 11 (Step ST130). In a case in which the transmission request is received, the screen generation unit 49 of the screen output control unit 48 generates screen data of the medical examination result display screen 95 illustrated in, for example, FIG. 15 (Step ST140). The screen data of the medical examination result display screen 95 is output to the screen transmission unit 50.

The screen transmission unit 50 transmits the screen data of the medical examination result display screen 95 to the owner terminal 11 which is the source of the transmission request (Step ST150). Step ST140 and Step ST150 are an example of a “screen output control step” according to the technology of the present disclosure.

In the owner terminal 11, the browser control unit 91 displays the medical examination result display screen 95 from the health management apparatus 10 on the display 34B. The owner OW browses the medical examination result display screen 95 and checks the medical examination results of the pet PT.

As illustrated in, for example, FIG. 15, the optical image OIM of the pet PT and the medical examination results are displayed on the medical examination result display screen 95. Since the optical image OIM of the pet PT owned by the user is displayed instead of a schema diagram prepared in advance, the owner OW who browses the medical examination result display screen 95 does not feel uncomfortable. Therefore, the owner OW can smoothly understand the medical examination results.

As illustrated in FIG. 8, the selection unit 47 selects an important item on the basis of the selection information 41. Specifically, the selection information 41 is information related to the normal ranges of the numerical values of the items of the medical examination and the selection unit 47 selects the item whose numerical value is out of the normal range as the important item. As illustrated in FIG. 15, the screen output control unit 48 displays the important item and other items in different display formats on the medical examination result display screen 95. Therefore, the owner OW can recognize the important item at a glance. The owner OW can easily understand the main points of the medical examination results, as compared to a case in which the important item and other items are displayed in the same display format without distinction. In addition, in a case in which the owner terminal 11 is a mobile terminal such as a smart phone, the display area of the display 34B is limited. Therefore, the display in which the main points are narrowed down is particularly effective.

In a case in which the important item has not been selected by the selection unit 47, that is, in a case in which the numerical values of all of the items are within the normal ranges, it is not necessary to display the results of the specimen examination.

The item whose numerical value is within the normal range and is close to the lower limit or the upper limit of the normal range may be selected as the important item. For example, the normal range of total protein is 6.5 g/dL to 8.0 g/dL. Even in a case in which the numerical value of total protein is in the range of 6.5 g/dL to 6.8 g/dL and 7.7 g/dL to 8.0 g/dL, total protein is selected as the important item. In addition, a level may be set to the important item according to the difference from the normal range. For example, a level indicating that treatment is required or a level indicating that treatment is not required, but caution is needed may be set to the important item. Then, the display format may be changed depending on the level.

As illustrated in FIG. 15, on the medical examination result display screen 95, the screen output control unit 48 displays the organs 60 so as to be superimposed on the optical image OIM and displays the annotation 105 indicating the organ 60 related to the important item on the optical image OIM. Therefore, even the owner OW who lacks medical knowledge immediately recognizes the organ 60 related to the important item. Therefore, it is possible to increase the understanding level of the owner OW on the medical examination results. In a case in which the item whose numerical value is out of the normal range, that is, the item having an abnormal numerical value is selected as the important item, the owner can understand what kind of organ 60 affects the abnormal numerical value. Therefore, it is possible to increase the understanding level of the owner OW on the medical examination results.

As illustrated in FIGS. 16 and 17, on the medical examination result display screen 95, the screen output control unit 48 displays the frame 80 indicating the imaging region in the image examination so as to be superimposed on a part of the body of the pet PT drawn in the optical image OIM. Therefore, the owner OW knows which part of the body of the pet PT is captured as the medical image displayed in the medical examination result display region 98 at a glance. Therefore, it is possible to increase the understanding level of the owner OW on the medical examination results.

### Second Embodiment

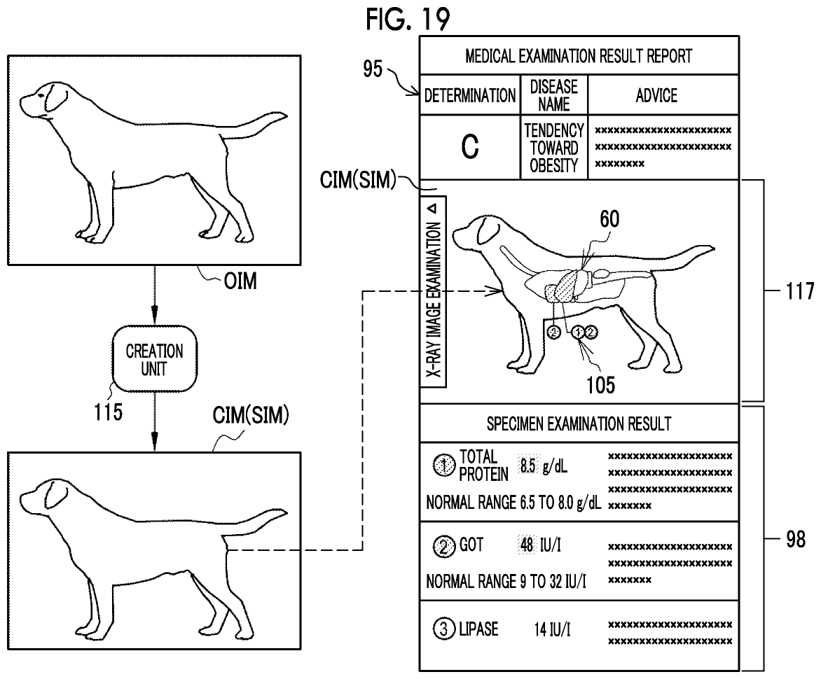

In a second embodiment illustrated in FIGS. 19 to 21, instead of or in addition to the optical image OIM, a schema diagram created on the basis of the optical image OIM is displayed on the medical examination result display screen 95.

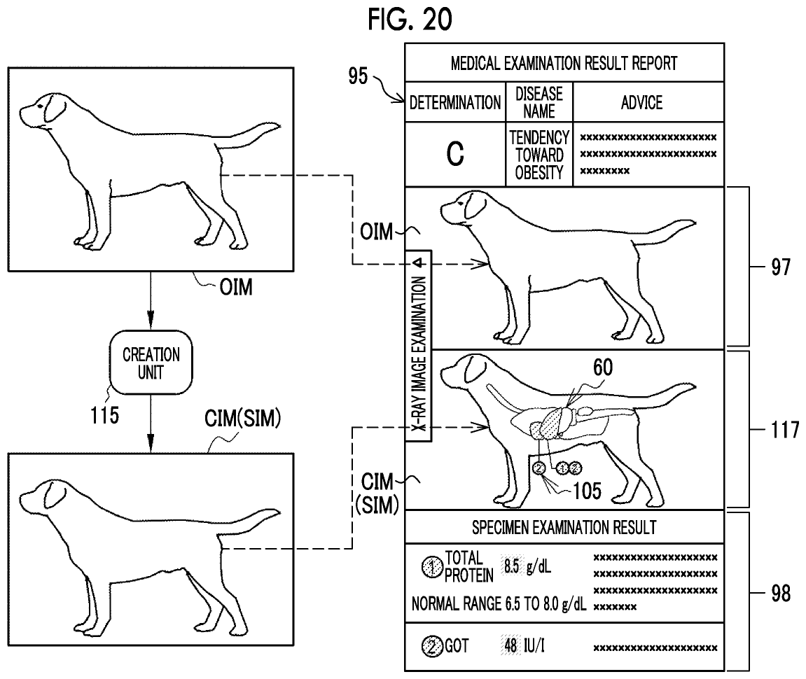

In FIGS. 19 and 20, in the second embodiment, the function of a creation unit 115 is added to the CPU 32A of the health management apparatus 10. The creation unit 115 creates a contour image CIM from the optical image OIM acquired by the first acquisition unit 45. For example, the creation unit 115 applies a schema diagram creation support AP described in <Internet URL; http://www.m-ss.com/mss/systems01.html> to the optical image OIM to create the contour image CIM. The creation unit 115 outputs the created contour image CIM as a schema diagram SIM to the screen generation unit 49.

As illustrated in FIG. 19, the screen generation unit 49 provides a schema diagram display region 117 in the medical examination result display screen 95, instead of the optical image display region 97. Then, the schema diagram SIM created by the creation unit 115 is displayed in the schema diagram display region 117. In addition, the screen generation unit 49 displays the organs 60 so as to be superimposed on the schema diagram SIM and displays the annotation 105 indicating the organ 60 related to the important item on the schema diagram SIM.

Alternatively, as illustrated in FIG. 20, the screen generation unit 49 provides the schema diagram display region 117 in the medical examination result display screen 95, in addition to the optical image display region 97. Then, similarly to the case illustrated in FIG. 19, the schema diagram SIM is displayed in the schema diagram display region 117. In addition, similarly to the case illustrated in FIG. 19, the screen generation unit 49 displays the organs 60 so as to be superimposed on the schema diagram SIM and displays the annotation 105 indicating the organ 60 related to the important item on the schema diagram SIM.

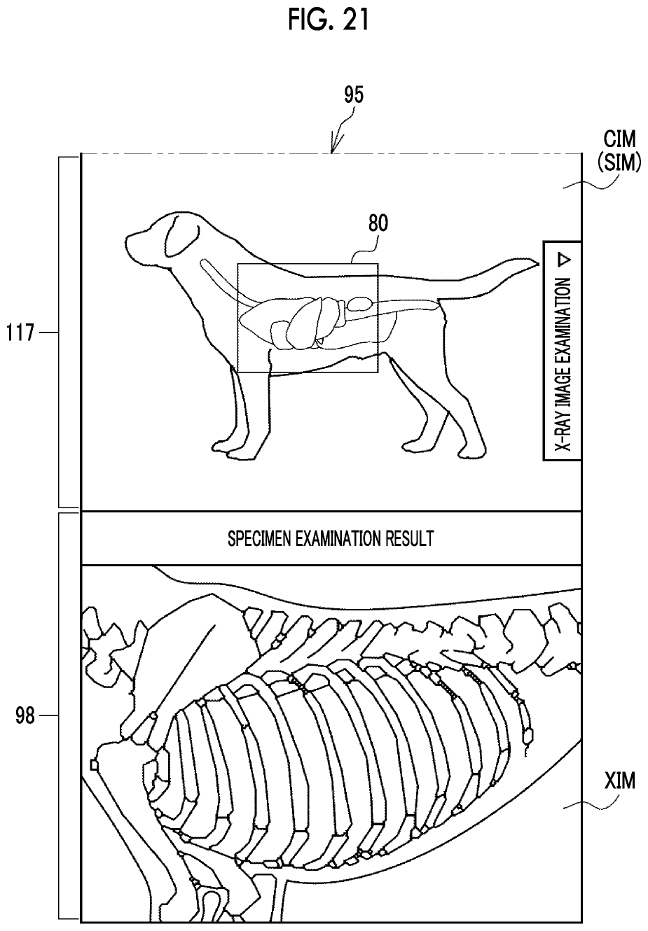

In both cases illustrated in FIGS. 19 and 20, as illustrated in FIG. 21, the screen generation unit 49 also displays the frame 80 so as to be superimposed on a part of the body of the pet PT drawn in the schema diagram SIM.

As such, in the second embodiment, the screen output control unit 48 displays the schema diagram SIM created on the basis of the optical image OIM on the medical examination result display screen 95, instead of or in addition to the optical image OIM. Therefore, similarly to the first embodiment, the owner OW can smoothly understand the medical examination results without feeling uncomfortable.

### Third Embodiment

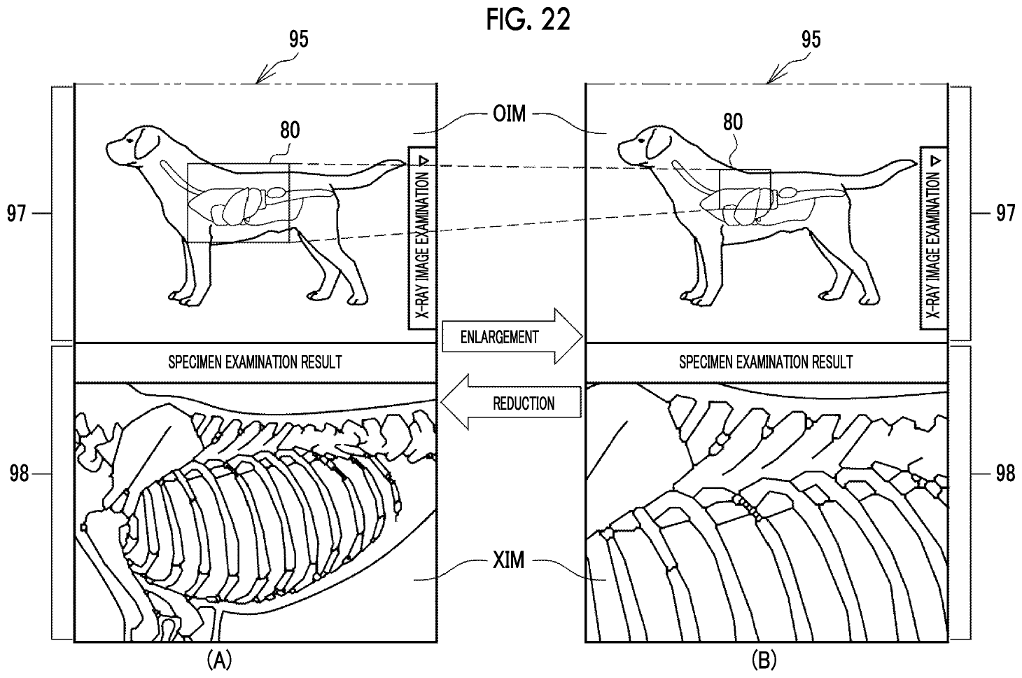

In a third embodiment illustrated in FIG. 22, the medical image obtained by the image examination is displayed so as be enlarged and reduced and the display size of the frame 80 is changed in operative association with the enlargement and reduction of the medical image.

In FIG. 22, the screen generation unit 49 displays an X-ray image XIM which is an example of the medical image in the medical examination result display region 98 so as be enlarged and reduced. In addition, the screen generation unit 49 changes the display size of the frame 80 which indicates an imaging region in the X-ray examination and is displayed so as to be superimposed on the optical image OIM in the optical image display region 97 in operative association with the enlargement and reduction of the X-ray image XIM. In this case, the medical examination result display region 98 is an example of “a region different from the frame” according to the technology of the present disclosure.

In a case in which the owner OW performs an enlargement operation from a state before enlargement illustrated in (A) of FIG. 22, the screen generation unit 49 enlarges the X-ray image XIM and displays the enlarged X-ray image XIM as illustrated in (B) of FIG. 22. With the enlargement and display of the X-ray image, the screen generation unit 49 reduces the display size of the frame 80. On the other hand, in a case in which the owner OW performs a reduction operation from the state illustrated in (B) of FIG. 22, the screen generation unit 49 reduces the X-ray image XIM and displays the reduced X-ray image XIM as illustrated in (A) of FIG. 22. Further, the screen generation unit 49 increases the display size of the frame 80 with the reduction and display of the X-ray image XIM. In addition, the enlargement operation is, for example, an operation of pinching out the X-ray image XIM. The reduction operation is, for example, an operation of pinching in the X-ray image XIM.

As such, in the third embodiment, the screen output control unit 48 displays the medical image obtained by the image examination in a region different from the frame 80 so as to be enlarged and reduced and changes the display size of the frame 80 in operative association with the enlargement and reduction of the medical image. Therefore, it is possible to enlarge and display a portion of the medical image that is desired to be viewed and to observe the portion in detail. In addition, it is possible to know which part of the body of the pet PT is captured as the medical image being observed, regardless of the enlargement and reduction of the medical image. Therefore, it is possible to increase the understanding level of the owner OW on the medical examination results.

### Fourth Embodiment

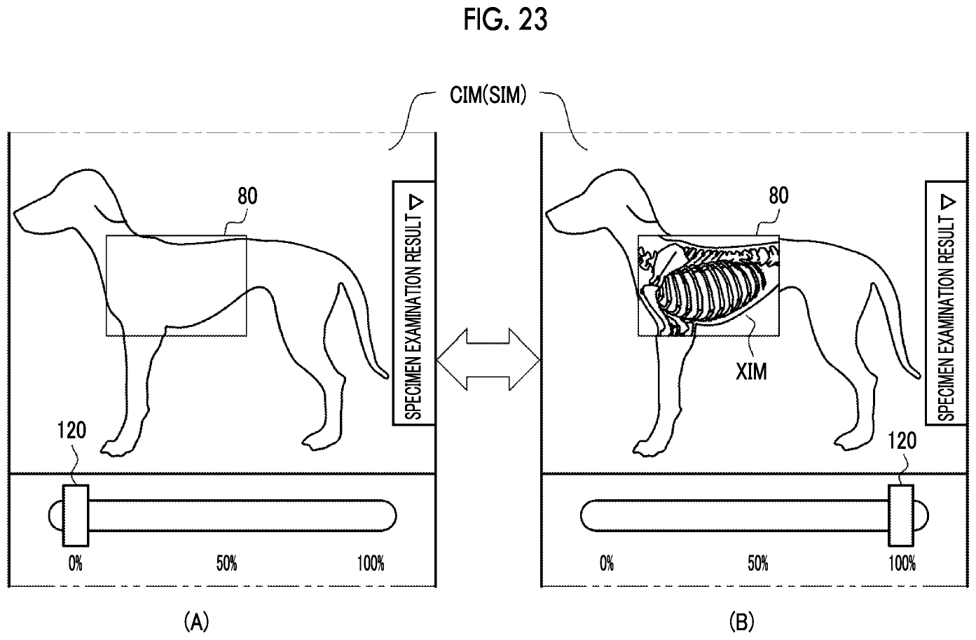

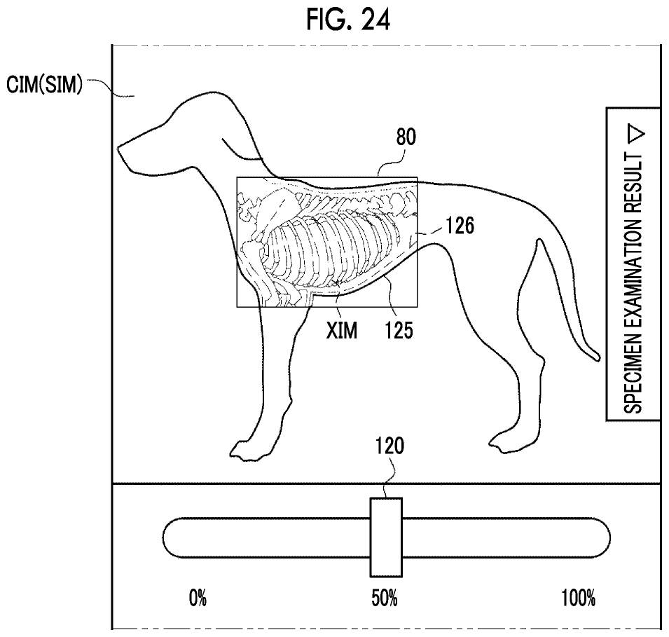

In a fourth embodiment illustrated in FIGS. 23 and 24, a medical image obtained by the image examination is displayed in the frame 80 and the transparency of at least one of the optical image OIM or the schema diagram SIM in the frame 80 is changed in response to the operation of the owner OW.

In FIG. 23, the screen generation unit 49 displays the frame 80 so as to be superimposed on the schema diagram SIM. The screen generation unit 49 displays the X-ray image XIM which is an example of the medical image in the frame 80 (see (B) of FIG. 23). In addition, as in the first embodiment, the frame 80 may be displayed so as to be superimposed on the optical image OIM, instead of the schema diagram SIM, and the X-ray image XIM may be displayed in the frame 80.

A transparency change bar 120 is provided below the schema diagram SIM. The transparency change bar 120 is a GUI for changing the transparency of the schema diagram SIM in the frame 80. The transparency change bar 120 is moved to a position between a transparency of 0% and a transparency of 100% in response to, for example, a swipe operation or a flick operation of the owner OW.

(A) of FIG. 23 illustrates a case in which the transparency change bar 120 is at a position corresponding to a transparency of 0%. In this case, the X-ray image XIM is not displayed, but only the schema diagram SIM is displayed in the frame 80. (B) of FIG. 23 illustrates a case in which the transparency change bar 120 is at a position corresponding to a transparency of 100%. In this case, contrary to the case illustrated in (A) of FIG. 23, the schema SIM is not displayed, but only the X-ray image XIM is displayed in the frame 80.

FIG. 24 illustrates a case in which the transparency change bar 120 is at a position corresponding to a transparency of 50%. In this case, both the schema diagram SIM and the X-ray image XIM are displayed in the frame 80. However, the X-ray image XIM is translucently displayed as represented by a dashed line. Therefore, a contour 125 of the pet PT by the schema diagram SIM and a contour 126 of the pet PT by the X-ray image XIM are displayed in the frame 80. The contour 125 by the schema diagram SIM is a body line including the hair of the pet PT. The contour 126 by the X-ray image XIM is a body line along the skin which does not include the hair of the pet PT.

As such, in the fourth embodiment, the screen output control unit 48 displays the medical image obtained by the image examination in the frame 80 and changes the transparency of at least one of the optical image OIM or the schema diagram SIM in the frame 80 in response to the operation of the owner OW. Therefore, it is possible to more intuitively understand which part of the body of the pet PT is captured as the medical image. As a result, it is possible to further increase the understanding level of the owner OW on the medical examination results.

As illustrated in FIG. 24, in the display aspect in which both the schema diagram SIM and the X-ray image XIM are displayed in the frame 80, the contour 125 that is a body line including the hair of the pet PT and the contour 126 that is a line along the skin of the pet PT are displayed. Therefore, the obesity of the pet PT can be checked by comparing the contour 125 with the contour 126. This effect is particularly effective for the breed of the pet PT which has long hair and whose skin lines are difficult to see. In a case in which the breed of pet PT having long hair is a dog, for example, the breed is Yorkshire Terrier, Papillon, or Maltese. In a case in which the breed of the pet PT having long hair is a cat, for example, the breed is Ragdolls, Himalayan, or Ragamuffin.

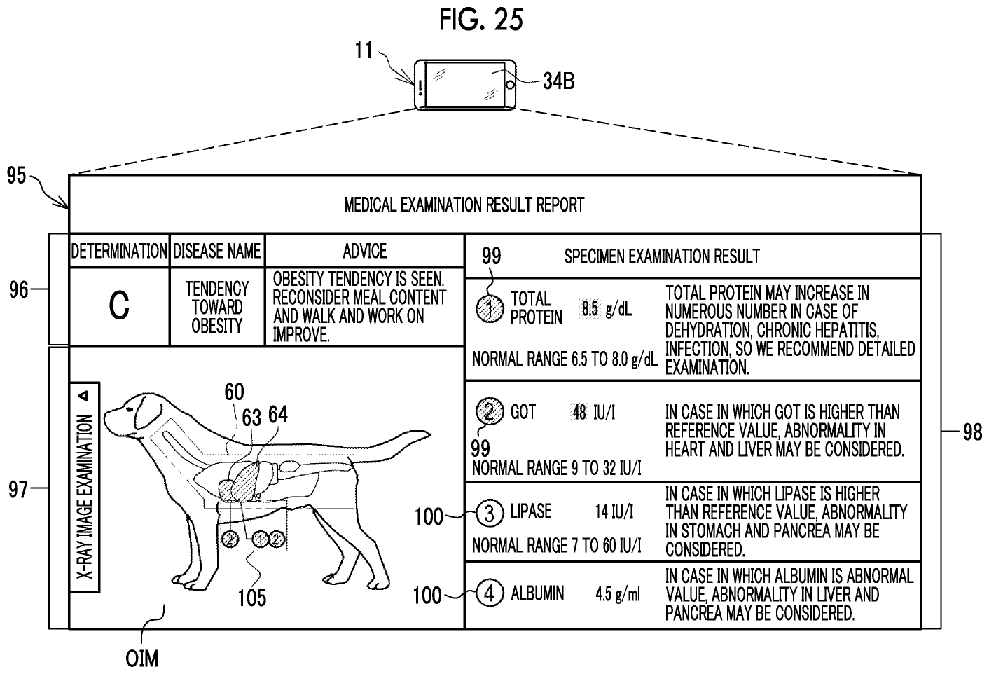

In each of the above-described embodiments, as illustrated in, for example, FIGS. 15 to 17, it is assumed that the display 34B of the owner terminal 11 is aligned in the vertical direction and the medical examination result display screen 95 in which the summary display region 96, the optical image display region 97, and the medical examination result display region 98 are arranged in the vertical direction is displayed. However, the present disclosure is not limited thereto. As in the medical examination result display screen 95 illustrated in FIG. 25, it is assumed that the display 34B of the owner terminal 11 is aligned in the horizontal direction and the medical examination result display region 98 may be laid out on the right side of the summary display region 96 and the optical image display region 97. It is preferable that the vertical medical examination result display screen 95 illustrated in FIGS. 15 to 17 and the horizontal medical examination result display screen 95 illustrated in FIG. 25 are switched according to the direction of the display 34B of the owner terminal 11.

The degree of obesity of the pet PT may be displayed so as to be superimposed on at least one of the optical image OIM or the schema diagram SIM. The degree of obesity of the pet PT is, for example, the rate of change in the measured value of the weight in the physical examination with respect to the ideal weight. The degree of obesity may be comprehensively calculated from, for example, body-fat percentage, neutral fat, total cholesterol, and a blood glucose level. Further, in a case in which the pet PT has a tendency toward obesity, a part that is estimated to be fat may be painted in red in at least one of the optical image OIM or the schema diagram SIM.

The animal species of the pet PT is not limited to the dogs and the cats described in each of the above-described embodiments. The animal species may be, for example, birds, rabbits, and hamsters.

The owner terminal 11 and/or the hospital terminal 12 may function as the health management apparatus 10.

The hardware configuration of the computer forming the health management apparatus 10 can be modified in various ways. For example, the health management apparatus 10 may be configured by a plurality of computers which are separated as hardware in order to improve processing capability and reliability. Specifically, the functions of the first acquisition unit 45, the second acquisition unit 46, and the selection unit 47 and the functions of the screen output control unit 48 are distributed to two server computers. In this case, the two server computers form the health management apparatus 10. Alternatively, the functions of the first acquisition unit 45 and the second acquisition unit 46 may be assigned to one acquisition unit.

As described above, the hardware configuration of the computer can be appropriately changed depending on the required performance such as processing capability, safety, and reliability. In addition to hardware, an AP, such the operation program 40, may be duplicated or may be dispersively stored in a plurality of storage devices in order to ensure safety and reliability.

In the above-described embodiments, for example, the following various processors can be used as the hardware structure of processing units performing various processes, such as the first acquisition unit 45, the second acquisition unit 46, the selection unit 47, the screen output control unit 48 (the screen generation unit 49 and the screen transmission unit 50), and the creation unit 115. The various processors include a CPU which is a general-purpose processor executing software to function as various processing units, a programmable logic device (PLD), such as a field programmable gate array (FPGA), which is a processor whose circuit configuration can be changed after manufacture, and/or a dedicated electric circuit, such as an application specific integrated circuit (ASIC), which is a processor having a dedicated circuit configuration designed to perform a specific process.

One processing unit may be configured by one of the various processors or a combination of two or more processors of the same type or different types (for example, a combination of a plurality of FPGAs and/or a combination of a CPU and an FPGA).

In addition, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As such, various processing units are configured by using one or more of the various processors as a hardware structure.

In addition, specifically, an electric circuit (circuitry) obtained by combining circuit elements, such as semiconductor elements, can be used as the hardware structure of the various processors.

It is possible to recognize the invention described in the following Supplementary Note 1 from the above description.

Supplementary Note 1

A health management apparatus comprises: a first acquisition processor that acquires medical examination results which are results of a medical examination of a pet; a second acquisition processor that acquires an optical image of the pet; and a screen output control processor that controls an output of a medical examination result display screen including the medical examination results and at least one of the optical image or a schema diagram created on the basis of the optical image.

In the technology according to the present disclosure, the above-described various embodiments and/or various modifications may be appropriately combined with each other. The technology according to the present disclosure is not limited to each of the above-described embodiments and can adopt various configurations without departing from the scope and spirit of the present disclosure. In addition, the technology according to the present disclosure may be applied to a program and a storage medium that non-transitorily stores the program.

The contents described and illustrated above are the detailed description of portions related to the technology according to the present disclosure and are merely examples of the technology according to the present disclosure. For example, the description of the configurations, the functions, the operations, and the effects is the description of an example of the configurations, functions, operations, and effects of a portion according to the technology of the present disclosure. Therefore, for the contents described and illustrated above, unnecessary portions may be deleted or new elements may be added or replaced without departing from the scope and spirit of the technology according to the present disclosure. In the contents described and illustrated above, the description of common technical knowledge that does not require any explanation in order to enable the implementation of the technology according to the present disclosure is omitted in order to avoid complications and facilitate the understanding of the portions related to the technology according to the present disclosure.

In the specification, “A and/or B” is synonymous with “at least one of A or B”. That is, “A and/or B” means A, B, or a combination of A and B. In the specification, the same concept as “A and/or B” is applied to a case in which three or more things are expressed by connecting them with “and/or”.

All of the documents, the patent applications, and the technical standards described in the specification are incorporated by reference to the same extent as the incorporation of each of the documents, the patent applications and the technical standards by reference is specifically and individually stated.

What is claimed is:

1. A health management apparatus comprising:
a first acquisition unit that acquires medical examination results which are results of a medical examination of a pet;
a second acquisition unit that acquires an optical image of the pet; and
a screen output control unit that controls an output of a medical examination result display screen including the medical examination results and at least one of the optical image or a schema diagram created on the basis of the optical image.

2. The health management apparatus according to claim 1, further comprising:
a selection unit that selects an important item from items of the medical examination on the basis of selection information stored in advance,
wherein the screen output control unit displays the important item in a display format different from a display format of other items on the medical examination result display screen.

3. The health management apparatus according to claim 2,
wherein the selection information is information related to normal ranges of numerical values of the items, and
the selection unit selects an item whose numerical value is out of the normal range as the important item.

4. The health management apparatus according to claim 2,
wherein the screen output control unit displays organs so as to be superimposed on least one of the optical image or the schema diagram and displays an annotation indicating an organ related to the important item on at least one of the optical image or the schema diagram.

5. The health management apparatus according to claim 1,
wherein the medical examination includes an image examination, and
the screen output control unit displays a frame indicating an imaging region in the image examination so as to be superimposed on a part of a body of the pet drawn in at least one of the optical image or the schema diagram.

6. The health management apparatus according to claim 5,
wherein the screen output control unit displays a medical image obtained by the image examination as the medical examination results in a region different from the frame so as to be enlarged and reduced and changes a display size of the frame in operative association with the enlargement and reduction of the medical image.

7. The health management apparatus according to claim 5,
wherein the screen output control unit displays a medical image obtained by the image examination as the medical examination results in the frame and changes transparency of at least one of the optical image or the schema diagram in the frame in response to an operation of a user.

8. A method for operating a health management apparatus, the method comprising:
a first acquisition step of acquiring medical examination results which are results of a medical examination of a pet;
a second acquisition step of acquiring an optical image of the pet; and
a screen output control step of controlling an output of a medical examination result display screen including the medical examination results and at least one of the optical image or a schema diagram created on the basis of the optical image.

9. A non-transitory computer-readable storage medium storing a program for operating a health management apparatus, the program causing a computer to function as:
a first acquisition unit that acquires medical examination results which are results of a medical examination of a pet;
a second acquisition unit that acquires an optical image of the pet; and
a screen output control unit that controls an output of a medical examination result display screen including the medical examination results and at least one of the optical image or a schema diagram created on the basis of the optical image.
